(19) United States
(12) Patent Application Publication
(10) Pub. No.: US 2012/0271114 A1
(43) Pub. Date: Oct. 25, 2012

(54) Choledochoilluminating Drainage Device

(75) Inventors: Yu-Chung Chang, Kaohsiung City (TW); Hsiang-Chen Chui, Taichung City (TW)

(73) Assignee: NATIONAL CHENG KUNG UNIVERSITY, Tainan City (TW)

(21) Appl. No.: 13/450,891

(22) Filed: Apr. 19, 2012

(30) Foreign Application Priority Data
Apr. 22, 2011 (TW) 100207198

(51) Int. Cl. A61B 1/06; A61M 27/00

(52) U.S. Cl. 600/182

(57) ABSTRACT

A choledochoilluminating drainage device is disclosed, which includes a drainage catheter and at least one optical fiber disposed in or on there. The optical fiber includes at least one light-emitting structure. While the drainage catheter is put into a duct in an organism, light can be emitted out from the light-emitting structure guided by the optical fiber disposed in or on the drainage catheter, passing through the walls of the drainage catheter and the organism's duct, thereby illuminating the organism's duct and the surrounding region.

### RELATED APPLICATIONS

This application claims priority to Taiwan Application Serial Number 100207198, filed Apr. 22, 2011, which is herein incorporated by reference.

### BACKGROUND

1. Field of Invention

The present invention relates to an endoscopic surgical device, and more particularly, to a choledochoilluminating drainage device.

2. Description of Related Art

The gallbladder concentrates and stores the bile juice secreted by the liver. When the fatty food enters the intestinal tract, the gallbladder contracts to release the bile juice into the small intestine for facilitating the digestion of fat. However, gallstones (cholelithiasis) are easily crystallized and formed when the bile juice becomes infected, accumulated or unbalanced. The gallstones block the bile duct and cause symptoms including the pain in the upper abdomen, vomiting, indigestion and so on often occur, and even more severe symptoms including fever and jaundice.

Slight cholelithiasis can be removed typically by the nonsurgical (noninvasive) treatment such as litholysis and lithotripsy. Its complication is very rare, but the recurrence rate is often more than fifty percent, resulting that repeated treatments and long-term drug administration are necessary to prevent recurrence. Therefore, such treatments are not widely applied in clinical practice.

With respect to severe cholelithiasis, it can be treated by the open cholecystectomy to excise the gallbladder. However, during the open cholecystectomy, an incision scar of 15 to 20 cm in length approximately is left in the right subcostal abdomen for perform the laparotomy and cholecystectomy. Moreover, the risk of such surgery is higher and needs longer post-operative caring period.

During the past several decades, laparoscopic cholecystectomy (LC) has become the standard treatment of choice for some symptomatic cholelithiasis diseases. It is widely applied in the treatment of cholelithiasis for it has benefits of minimal invasiveness.

In brief, laparoscopic cholecystectomy is simple and safe laparoscopic procedure performed for 30 minutes to one and half hours approximately. In this procedure, three or four incision wounds with 5-10 mm diameter can be introduced into the abdomen through trocars (hollow tubes with a seal to keep 2-5 liters of the CO₂ from leaking). Although LC operative techniques are more mature for most cholelithiasis patients currently, some patients with existing pulmonary disorders may not tolerate pneumoperitoneum (gas in the abdominal cavity), resulting in a need for conversion to open surgery after the initial attempt at laparoscopic approach. In addition, dense adhesions with the stomach, duodenum, the large intestine or the omentum from previously chronic inflammation of the gallbladder may spend longer time in LC operation and are considered to the conventional open cholecytectomy.

A successful LC depends on the skillful operation of a surgeon, the knowledge and awareness of the bile duct system and the surrounding organs, and careful selection of patients who are suitable to perform the LC. There are many advantages existing in the LC, but accidental bile duct injuries still happen for the following reasons. First of all, the surgeon has wrong determination or less experience. Moreover, inflammation of gall bladder, Calot's triangle and hepatoduodenal ligament areas ranges from edema, swelling and fibrosis. Those reasons easily cause erroneous judgment or excision of the common bile duct to the common hepatic duct, resulting in the accidental bile duct injuries. For example, it obviously increases 2˜3 folds incidence of iatrogenic common bile duct (CBD) injury rate (from 01.˜0.2% to 0.4˜0.6%) when compare with the era of open cholecystectomy.

Injuries to the CBD are generally expensive and difficult to repair and may lead to an irreversible tragedy of the patient. CBD injury may even cause end stage liver disease and necessitate liver transplantation, mortality during the waiting, early postoperative, and late postoperative periods is 45%.

Many surgeons have argued that the best way to avoid errors related to the misperception of structures is by the practice of routine intra-operative cholangiography (IOC). However, others have found that routine IOC is not necessary to avoid CBD injuries.

Therefore, it is necessary to provide a surgical instrument for decreasing the risk of accidental duct injuries during the gallbladder excision surgery.

### SUMMARY

A choledochoilluminating drainage device is provided. The choledochoilluminating drainage device includes at least one optical fiber disposed in or on a tube wall of a drainage catheter, and the optical fiber includes at least one light-emitting structure. When the drainage catheter is put into a duct or a to-be-treated portion in an organism, the optical fiber transmits a light of a light source to emit out from the light-emitting structure and to pass through the tube wall of the drainage catheter and the organism's duct, thereby illuminating the duct (or the to-be-treated portion) and the surrounding region.

Moreover, a choledochoilluminating drainage device is provided. The choledochoilluminating drainage device includes the aforementioned choledochoilluminating drainage catheter and an irrigation catheter both of which are received in an inner tubular space of an outer cannula. When the drainage catheter and the irrigation catheter are both put into a duct or a to-be-treated portion in an organism, the optical fiber transmits a light of a light source to emit out from the light-emitting structure and to pass through the tube wall of the drainage catheter and the duct of the organism, thereby illuminating the duct (or the to-be-treated portion) and the surrounding region.

Accordingly, the invention provides a choledochoilluminating drainage device is provided. In an embodiment, the choledochoilluminating drainage device comprises a drainage catheter and at least one optical fiber. The drainage catheter has a drainage end. The optical fiber is disposed in or on a tube wall of the drainage catheter, in which the optical fiber includes at least one light-emitting structure, and the light-emitting structure comprises an emitting end and a plurality of emitting sides. In an example, the emitting end is disposed at an end of the optical fiber and adjacent to the drainage end. In another example, the emitting sides are disposed on an external side of the optical fiber and adjacent to the emitting end. When the drainage catheter is put into a duct or a to-be-treated portion in an organism, the optical fiber transmits a light of a light source to emit out from the light-emitting structure and to pass through the tube wall of the drainage catheter and the duct of the organism, thereby illuminating the duct (or the to-be-treated portion) and the surrounding region.

According to an embodiment of the invention, the aforementioned optical fiber is disposed on an internal side, an external side or in the tube wall of the drainage catheter.

According to an embodiment of the invention, a diameter of the optical fiber around the emitting sides is less than an original diameter of the optical fiber. In an example, a plurality of microstructures are disposed on the emitting sides.

According to an embodiment of the invention, the aforementioned light source is disposed at another end of the optical fiber, and the light has a wavelength of 520 nm to 540 nm. In an example, a luminance of the light emitted from the emitting sides is 10 W/mm² to 20 W/mm².

According to an embodiment of the invention, the aforementioned duct is a common bile duct or a ureter.

With application of the choledochoilluminating drainage device, while the drainage catheter is put into a duct in an organism, light can be emitted out from the light-emitting structure guided by the optical fiber disposed in or on the drainage catheter, passing through the walls of the drainage catheter and the organism's duct, thereby illuminating the duct (or the to-be-treated portion) and the surrounding region. Therefore, the device is able to decrease the risk of accidental duct injuries during the laparo-endoscopic surgery.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 depicts a cross-sectional diagram of the choledochoilluminating drainage device according to an embodiment of the present invention.

FIG. 2 depicts a partial diagram of the light-emitting structure of the optical fiber according to an embodiment of the present invention.

FIGS. 3A to 3C depict partial diagrams of the light-emitting structure of the optical fiber according to several embodiments of the present invention.

FIG. 4 depicts a partial diagram of the choledochoilluminating drainage device according to another embodiment of the present invention.

FIG. 5 depicts an operation diagram of the choledochoilluminating drainage device during the laparoscopic cholecystectomy (LC) according to an embodiment of the present invention.

FIG. 6 depicts an operation diagram of the choledochoilluminating drainage device during the LC according to another embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

As aforementioned, the invention relates to a choledochoilluminating drainage device, which includes at least one optical fiber disposed in or on a tube wall of a drainage catheter, and the optical fiber includes at least one light-emitting structure. When the drainage catheter is put into a duct or a to-be-treated portion in an organism, the optical fiber transmits a light of a light source to emit out from the light-emitting structure and to pass through the tube wall of the drainage catheter and the duct of the organism, thereby illuminating the duct (or the to-be-treated portion) and the surrounding region.

The “duct in the organism” as discussed hereinafter is referred to a common bile duct, a ureter or surrounding ducts. During the endoscopic surgery such as laparoscopic cholecytectomy (LC) for treating the patient who suffers gallbladder-related diseases, the accumulated bile juice must be drained out by using endoscopic nasobiliary drainage (ENBD) or endoscopic retrograde biliary drainage (ERBD). However, as aforementioned, the common bile duct injuries often found due to various improper treatments may cause irreversible tragedies to the patients. For the purpose of reducing such risk of the endoscopic surgery, the present invention provides a choledochoilluminating drainage device for guiding light to passing through the organism's duct and to illuminate the duct (or the to-be-treated portion) and the surrounding region. Therefore, the choledochoilluminating drainage device can further prevent the accidental injuries of the common bile duct or the ureter during the endoscopic surgery (e.g. LC).

Structure of Choledochoilluminating Drainage Device

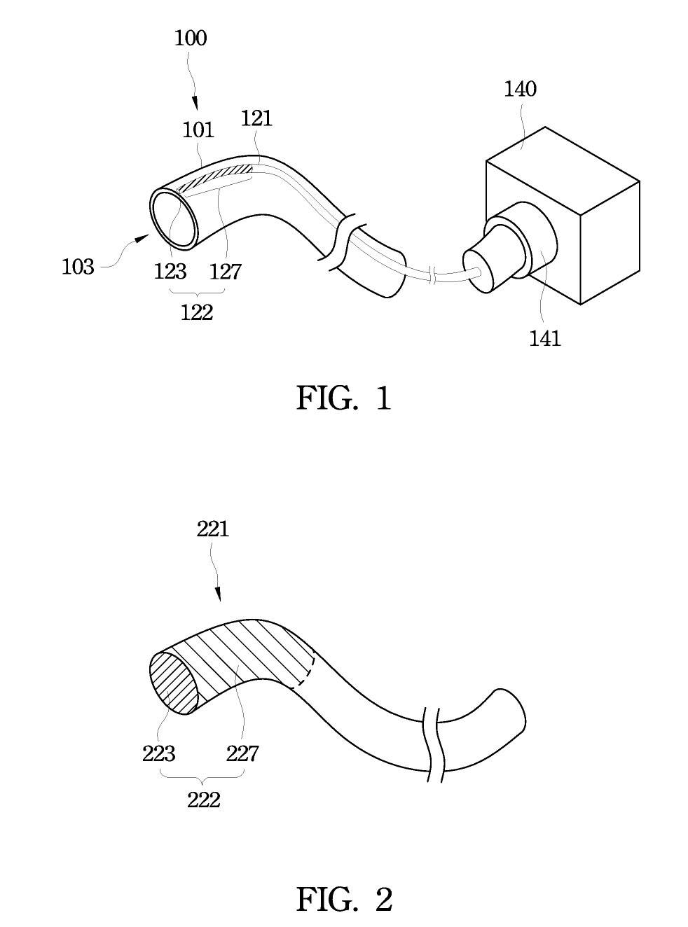

Reference is made to FIG. 1, which depicts a cross-sectional diagram of the choledochoilluminating drainage device according to an embodiment of the present invention. In an embodiment, the choledochoilluminating drainage device 100 comprises a drainage catheter 101 and at least one optical fiber 121. In this embodiment, the drainage catheter 101 may be exemplified as commercially available products such as endoscopic nasal biliary drainage (ENBD; Wilson-Cook Medical, Inc., U.S.A.) or other functionally equivalent catheters. The length and outer diameter of the drainage catheter 101 depends on the actual requirements rather than being limited thereto. However, the drainage catheter 101 must be put through oral or nasal passage, gallbladder or bladder and brought deeply to the common bile duct or the ureter, the length of the drainage catheter 101 is preferably equal to one meter or more, such as one meter to three meters, and its outer diameter is less than the averaged width of the common bile duct or the ureter, preferably one micrometer (mm) to 10 mm, and more preferably 3 mm to 5 mm. In other embodiments, the drainage catheter 101 may optionally have several holes (unshown) near the drainage end at the front tube wall, so as to drain the body fluid out and to decrease the possibility of the calculus obstruction.

In an embodiment, the optical fiber 121 may be an optical fiber pigtail such as FC type pigtail fiber (single mode, cord/cladding=9/125 μm), and the optical fiber includes a material of polyvinylchloride (PVC) or other known materials. Moreover, the optical fiber 121 may disposed on an internal side, an external side or in the tube wall of the drainage catheter by using the prior fixation ways such as laser melting, UV curing or the like.

The light source 140, for example, green light laser, is disposed at another end of the optical fiber through an adapter device 141, so as to generate the light with a wavelength of 520 nm to 540 nm or approximately 530 nm. The illuminance and luminance of the light-emitting structure 122 must be less than the photodamage threshold value, so that the light-emitting structure 122 of the optical fiber 121 provides illumination but does not induce photodamage to the organism's tissue. For example, an illuminance of the light emitted from the emitting end 123 is 1 mW to 10 mW, and a luminance of the light emitted from the emitting sides is 10 W/mm² to 20 W/mm².

One of the characteristics of the present invention is that, when the drainage catheter is put into a duct or a to-be-treated portion in an organism, the optical fiber transmits a light of a light source to emit out from the light-emitting structure and to pass through the tube wall of the drainage catheter and the duct of the organism, thereby illuminating the duct (or the to-be-treated portion) and the surrounding region. Reference is made to FIG. 2, which depicts a partial diagram of the light-emitting structure of the optical fiber according to an embodiment of the present invention. In FIG. 2, the light-emitting structure 222 may include an emitting end 223 and a plurality of emitting sides 227. The emitting end 223 is typically disposed at an end of the optical fiber 221 and adjacent to the drainage end 103 of the drainage catheter 101 of FIG. 1. The emitting sides 227 are disposed on an external side of a body of the optical fiber 221 and adjacent to the emitting end or not. In general, as the optical fiber 221 has no emitting sides 227 formed thereon, the light generated from the light source 140 is kept and transmitted inside the optical fiber 221 by total internal reflection except being emitting out from the emitting end 223. However, the emitting sides 227 can destruct the total internal reflection inside the optical fiber 221, so as to allow light transmitted inside the optical fiber 221 to be also emitted from the emitting sides 227.

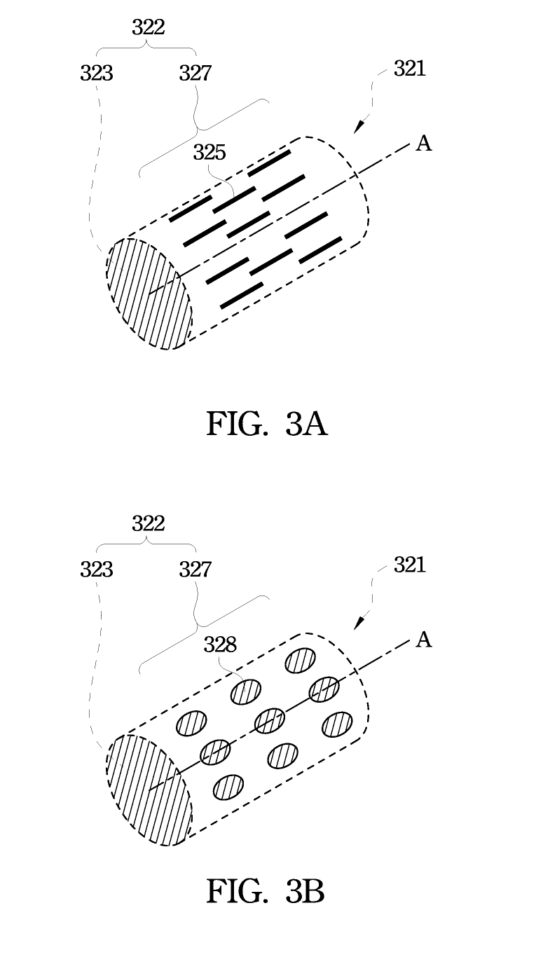

In addition, in order to enhancing the emitting efficiency of the optical fiber, the light-emitting structure on the sides of the optical fiber can have a plurality of microstructures formed thereon. Reference is made to FIGS. 3A to 3C, which depict partial diagrams of the light-emitting structure of the optical fiber according to several embodiments of the present invention. In an example, the light fiber 321 can have a plurality of microstructures such as nicks (for example, the nick 325 shown in the FIG. 3A) or indentations (for example, the indentation 328 shown in the FIG. 3B) formed on the emitting sides, so as to destruct the total internal reflection inside the optical fiber 321 and to facilitate the emitting efficiency of the sides of the optical fiber 321. The aforementioned nicks or indentations may have at least one size respectively, arranged regularly or irregularly. However, the sizes or specifications of the nicks or indentations are well known by the artisan in the art rather than being described in detail.

In another example, the diameter of the optical fiber 321 (for example, the diameter D₁ shown in the FIG. 3C) around the emitting sides 327 can be less than an original diameter of the optical fiber 321 (for example, the diameter D₂ shown in the FIG. 3C), so as to destruct the total internal reflection inside the optical fiber 321 and to facilitate the emitting efficiency of the sides of the optical fiber 321.

It should be supplemented that, in other examples, one or more optical fibers can be arranged arbitrarily and disposed on an internal side, an external side or in the tube wall of the drainage catheter (unshown). However, various modifications of the arrangements of the optical fibers can be disposed in various arrangements that are well known by the artisan in the art rather than being described in detail.

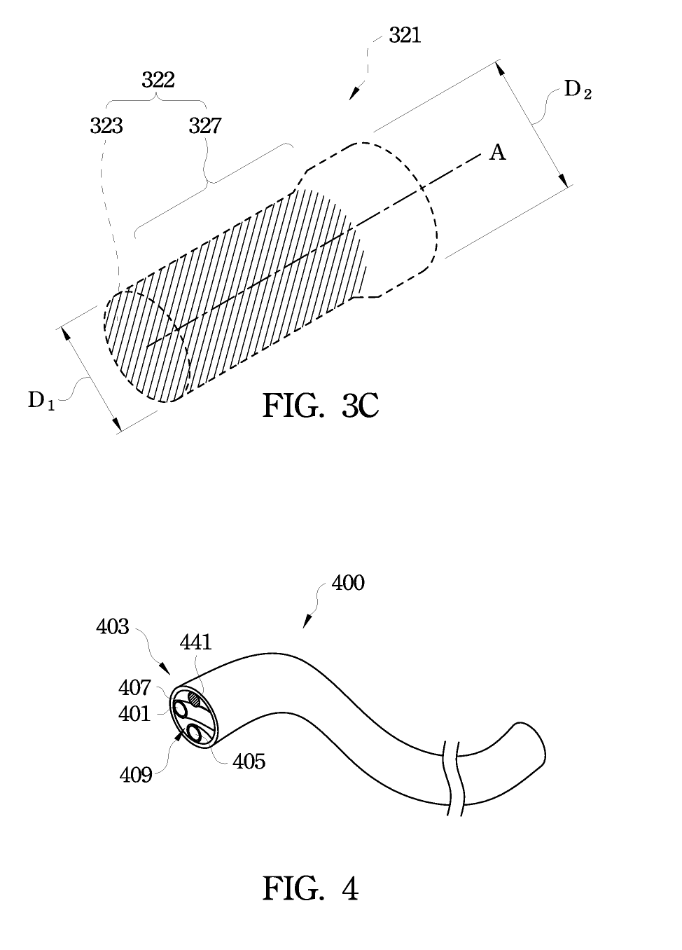

Reference is made to FIG. 4, which depicts a partial diagram of the choledochoilluminating drainage device according to another embodiment of the present invention. In an embodiment, the choledochoilluminating drainage device 400 further comprises a drainage catheter 401 and an irrigation catheter 405 both of which are received in an outer cannula 407.

The aforementioned outer cannula 407 is exemplified as a trocar for the conventional endoscopic surgery. In an embodiment, an inner tubular space 409 of the outer cannula 407 may include a catheter 401 and a catheter 405. The catheter 401 is exemplified as the aforementioned choledochoilluminating drainage catheter 101 of FIG. 1, and the catheter 405 is exemplified as an irrigation catheter. The catheter 401 and the catheter 405 may be commercially available products such as endoscopic nasal biliary drainages (ENBD; Wilson-Cook Medical, Inc., U.S.A.) or other commercially available and functionally comparable products.

In other embodiments, a video capture device 441 may optionally received in the inner tubular space 409 of the outer cannula 407 of the choledochoilluminating drainage device 400, as shown in FIG. 4. The video capture device 441 can be a prior device such as a complementary metal-oxide semiconductor (CMOS), a charge coupled device (CCD) or other functionally comparable products. The video capture device 441 is also connected to a video converting device (unshown), so as to convert the video to digitalized video data for being evaluated by an operator.

Operation of Choledochoilluminating Drainage Device

During operation, the catheter 401 can be put alone or with another catheter 405 into the organism's duct (for example, the common bile duct or the ureter). When irrigating the ducts in a subject (for example, human), the catheter 401 can connect to the given duct and drain the body fluid (for example, the accumulated bile juice) out through a drainage end 403 of the catheter 401 and/or the holes on the front tube wall of the catheter 401 near the drainage end 403. A washing solution (for example, the saline solution) is infused into the duct from the drainage end 403 of the catheter 405 for irrigating the duct, and then the waste solution is drained out through the catheter 401.

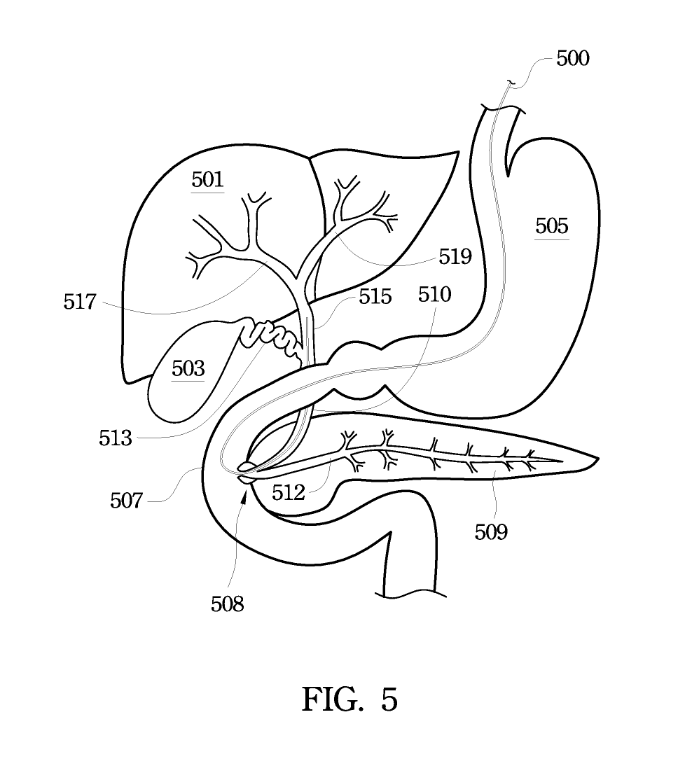

Reference is made to FIG. 5, which depicts an operation diagram of the choledochoilluminating drainage device during the laparoscopic cholecystectomy (LC) according to an embodiment of the present invention. Hereinafter, the LC is merely exemplified to illustrate how the choledochoilluminating drainage device is operated and how it is positioned and confirmed by X-ray developing apparatus. The choledochoilluminating drainage device 500 such as the choledochoilluminating drainage device 100 of FIG. 1 can be put into the duct from mouth or nose (shown) of the organism (for example, human beings or animals) along the path of ERBD. After passing through the stomach 505, the choledochoilluminating drainage device 500 (such as the choledochoilluminating drainage device 100 of FIG. 1) is retrograde entered into the common bile duct 510 from an opening of the sphincter of Oddi 508 in the middle of the duodenum 507. Also, the choledochoilluminating drainage device 500 should carefully prevent from entering into the pancreatic duct 512 of the pancreas 509.

The choledochoilluminating drainage device 500 is passed by the opening of the biliary cystic duct 513 and then it the common hepatic duct 515. In the meanwhile, the choledochoilluminating drainage device 500 emits light of an external light source (unshown) out from the light-emitting structure (for example, the light-emitting structure 122 of FIG. 1) guided by the optical fiber (for example, the optical fiber 121 of FIG. 1) disposed on the drainage catheter, passing through the common bile duct 510 and the common hepatic duct 515, thereby illuminating and clearly showing locations of the common bile duct 510, the common hepatic duct 515 and the surrounding region.

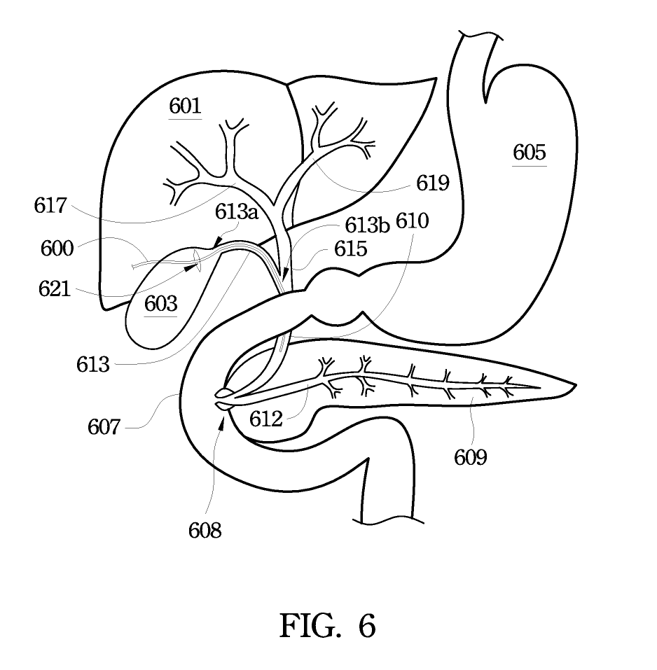

Reference is made to FIG. 6, which depicts an operation diagram of the choledochoilluminating drainage device during the LC according to another embodiment of the present invention. In this embodiment, the location of the common bile duct can be accurately and visually determined rather than X-ray developing apparatus.

At first, an opening 621 is formed by cutting the gall bladder 603 distal to the biliary cystic duct 613 during the LC, so that the choledochoilluminating drainage device 600 can pass through the opening 621 and the end of distal orifice of the biliary cystic duct 613 that is near the gall bladder 603, entering into the common bile duct 610.

Since the locations of the common bile duct 510 or the common hepatic duct 515 are shown clearly by the light guided by the choledochoilluminating drainage device 500 of FIG. 5 (or the choledochoilluminating drainage device 600 of FIG. 6), the biliary cystic duct 513 (or the biliary cystic duct 613) and the gall bladder 503 (the gall bladder 603) can be excised accurately, and the risk such as personally erroneous judgment or excision of the common bile duct 510 (or the common bile duct 610) or the common hepatic duct 515 (the common hepatic duct 615) can be avoided effectively. Thus, the choledochoilluminating drainage device 500 (or the choledochoilluminating drainage device 600) successfully prevents from the personally erroneous excision of the common bile duct 510 (or the common bile duct 610) or the common hepatic duct 515 (the common hepatic duct 615), the bile juice secreted by the liver can still drain into the duodenum through the right duct 517 or left hepatic duct 519 of the liver 501 (or the right duct 617 or left hepatic duct 619 of the liver 601) to the common bile duct 510 (or the common bile duct 610) without influencing fat digestion after the aforementioned surgery.

Moreover, the choledochoilluminating drainage device 500 provides the drainage catheter (for example, the drainage catheter 101) can drain the accumulated bile juice out and/or wash the common bile duct 510 and the common hepatic duct 515.

It should be supplemented that, although the present invention has been described in considerable detail with reference to certain embodiments thereof, other embodiments are possibly applied in the laparoscopic cholecystectomy. It is necessarily supplemented that, specific devices, specific components, specific structures, specific to-be-treated portions or specific apparatuses are employed as exemplary embodiments for clarifying the choledochoilluminating drainage device of the present invention and application on the laparoscopic cholecystectomy. However, as is understood by a person skilled in the art, other devices, other components, other structures, other to-be-treated portions or other apparatuses can be also employed in the choledochoilluminating drainage device of the present invention and application on other endoscopic surgeries, rather than being limited thereto. For instance, the choledochoilluminating drainage device of the present invention can be also applied on other endoscopic surgeries such as the pelvic surgery, thereby effectively prevent from the tragedy of erroneous excision of the ureter.

It is worth mentioning that, in addition to applications of Illumination and drainage, the choledochoilluminating drainage device is also applied on the photo dynamic therapy, in which the choledochoilluminating drainage device is put in the common bile duct, the ureter and the to-be-treated portion for a long-term period (more than one hour). In some embodiments, the to-be-treated portion (for example, cancer tissue) can be labeled by the photosensitizer (also called as light-activated drug) such as 5-aminolevulinic acid (5-ALA) or other photosensitizers. After the photosensitizer is attached to the to-be-treated portion, the choledochoilluminating drainage device guides a given wavelength of the light. The light can convert the photosensitizer from a ground state to an excited state, and the wavelength of the light depends on the selected photosensitizer. After the photosensitizer absorbs the light energy, it releases energy, singlet oxygen and free radical from the ground state to the excited state, thereby killing cancer cells of the to-be-treated portion and achieving the treatment effect.

In other embodiments, the choledochoilluminating drainage device is also combined with metal nanoparticles to perform the photodynamic therapy. When the metal nanoparticles (for example, gold nanoparticles) are labeled to the to-be-treated portion (for example, the cancer tissue), the choledochoilluminating drainage device guides the light with the given wavelength on the metal nanoparticles. And then, the surface plasma phenomenon of the metal nanoparticles can generate heat for achieving the treatment effect.

In addition, the choledochoilluminating drainage device is combined with both of the photosensitizer and the metal nanoparticles. The surface plasma phenomenon and heat of the metal nanoparticles after absorbing light can further enhance the cell toxicity of the photosensitizer, thereby achieving the treatment effect.

According to the embodiments of the present invention, the aforementioned choledochoilluminating drainage device advantageously uses the drainage catheter that can be put into a duct in an organism before or during the endoscopic surgery. While the drainage catheter is put into a duct in an organism, light can be emitted out from the light-emitting structure guided by the optical fiber disposed in or on the drainage catheter, passing through the walls of the drainage catheter and the organism's duct, thereby illuminating the duct (or the to-be-treated portion) and the surrounding region for the purpose of illumination or photodynamic therapy. Therefore, the device can mainly decrease the risk of accidental duct injuries during the endoscopic surgery.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A choledochoilluminating drainage device, comprising:
a drainage catheter, wherein the drainage catheter has a drainage end; and
at least one optical fiber disposed in or on a tube wall of the drainage catheter, wherein the optical fiber comprises at least one light-emitting structure, and the light-emitting structure comprises:
an emitting end disposed at an end of the optical fiber and adjacent to the drainage end; and
a plurality of emitting sides disposed on an external side of the optical fiber and adjacent to the emitting end, and
when the drainage catheter is put into a duct or a to-be-treated portion in an organism, the optical fiber transmits a light of a light source to emit out from the light-emitting structure and to pass through the tube wall of the drainage catheter and the duct of the organism, thereby illuminating the duct or the to-be treated portion and a surrounding region.

2. The choledochoilluminating drainage device of claim 1, wherein the optical fiber is disposed on an internal side, an external side or in the tube wall of the drainage catheter.

3. The choledochoilluminating drainage device of claim 1, wherein the optical fiber includes a material of polyvinylchloride (PVC).

4. The choledochoilluminating drainage device of claim 1, wherein the light source is disposed at another end of the optical fiber, and the light has a wavelength of 520 nm to 540 nm.

5. The choledochoilluminating drainage device of claim 1, wherein a diameter of the optical fiber around the emitting sides is less than an original diameter of the optical fiber.

6. The choledochoilluminating drainage device of claim 1, wherein a plurality of microstructures are disposed on the emitting sides, the microstructures are arranged regularly or irregularly, and the microstructures has at least one size.

7. The choledochoilluminating drainage device of claim 6, wherein the microstructures are a plurality of nicks or indentations.

8. The choledochoilluminating drainage device of claim 1, wherein an illuminance of the light emitted from the emitting end is 1 mW to 10 mW.

9. The choledochoilluminating drainage device of claim 1, wherein a luminance of the light emitted from the emitting sides is 10 W/mm² to 20 W/mm².

10. The choledochoilluminating drainage device of claim 1, wherein the duct is a common bile duct or a ureter.

11. The choledochoilluminating drainage device of claim 1, wherein the to-be-treated portion is a tumor tissue.

12. The choledochoilluminating drainage device of claim 1, wherein the to-be-treated portion is labeled by a photosensitive substance and/or metal nanoparticles.
